SEQUENCE LISTING (3 sequences)

SEQ ID NO: 1 - DNA, 24 bp, Artificial Sequence
  Forward primer for PCR amplification of osteopontin
agtgatttgcttttgcctgtttgg

SEQ ID NO: 2 - DNA, 23 bp, Artificial Sequence
  Reverse primer for PCR amplification of osteopontin
aggctgtaaagcttctcctctga

SEQ ID NO: 3 - DNA, 16 bp, Artificial Sequence
  Probe for detection of osteopontin nucleic acid
ccctcccggtgaaagt

(19) United States
(12) Patent Application Publication
(10) Pub. No.: US 2010/0105046 A1
(43) Pub. Date: Apr. 29, 2010

(54) METHODS OF DIAGNOSING CARDIOVASCULAR DISEASE

(75) Inventors: Stephen E. Epstein, Rockville, MD (US); Mary Susan Burnett, Fairfax, VA (US)

Correspondence Address: WOLF GREENFIELD & SACKS, P.C., 600 ATLANTIC AVENUE, BOSTON, MA, 02210-2206, US

(73) Assignee: MedStar Health, Inc., Columbia, MD (US)

(21) Appl. No.: 12/535,614

(22) Filed: Aug. 4, 2009

Related U.S. Application Data
(63) Continuation of application No. 11/078,743, filed on Mar. 11, 2005, now abandoned.
(60) Provisional application No. 60/557,801, filed on Mar. 29, 2004.

(51) Int. Cl. C12Q 1/68

(52) U.S. Cl. 435/6

(57) ABSTRACT

The invention relates to predicting which individuals are at risk of developing atherosclerotic vascular disease, and once having disease, which individuals are at risk of experiencing plaque rupture which, depending on the site of the plaque, could produce myocardial infarction, stroke, critical limb ischemia, or other vascular event. The invention further relates to methods of diagnosing and aiding in the diagnosis of vascular conditions such as atherosclerosis, premature coronary artery disease and coronary artery disease, by detecting a resistin gene product in an individual. The invention further relates to methods of predicting, and aiding in predicting, the likelihood that an individual will experience a vascular event, such as but not limited to, a myocardial infarction, acute coronary syndrome, stroke, transient ischemic attack (TIA), or critical limb ischemia.

### CROSS-REFERENCE TO RELATED APPLICATIONS

This application claims the benefit of the filing date of U.S. application Ser. No. 60/557,801, filed Mar. 29, 2004, entitled “METHODS OF DIAGNOSING, PREVENTING AND TREATING CARDIOVASCULAR DISEASE.” The entire teachings of the referenced application are incorporated by reference herein.

### BACKGROUND OF THE INVENTION

Coronary artery disease is a multifactor disease that results in the deposition of athermanous plaque and progressive luminal narrowing of the arteries that supply the heart muscle. This plaque consists of a mixture of inflammatory and immune cells, fibrous tissue, and fatty material such as low-density lipoprotein cholesterol (LDL-C) and modifications thereof, and α-lipoprotein. The luminal narrowing can occur slowly which, depending on the severity of obstruction, results in reduced ability to deliver oxygen and nutrients to the heart muscle, producing such vascular events as myocardial infarction, angina, unstable angina, and sudden ischemic death as heart failure. Though occlusion can progress slowly, blood supply often is cut off suddenly when a portion of the built-up arterial plaque ruptures and exposes the rapidly moving blood within the arterial lumen to the contents of the plaque; thrombus develops within the lumen to block the artery temporarily or permanently, often leading to death of that portion of myocardial tissue supplied by the obstructed artery. Depending on the volume of muscle distal to the blockage during such an attack, the patient may develop heart failure due to weakening the heart muscle supplied by the obstructed artery, or may die.

Two key conundrums relating to the ability to diagnosis patients who will soon, or in the future, develop plaque rupture with its dangerous consequences, is that plaque rupture most often develops in a plaque that is not producing critical stenosis just prior to rupture. This is reflected in part by the fact that in 40-60% of the individuals who are eventually diagnosed as having coronary artery disease, myocardial infarction is the first presentation of disease. Hence, standard diagnostic tests, which rely on detecting lesions that cause significant impairment of flow to the myocardium (typically during exercise), may be negative just prior to a fatal rupture. Such tests include exercise stress testing (“EST”) using ECG monitoring, Thallium-201 scintigraphy, exercise echocardiography, and ambulatory ECG (Holter monitoring). In addition, many patients have significant lesions and may actually have abnormalities in the above tests, but none of the lesions are “vulnerable”—that is, they do not have the typical features of a plaque at risk of rupture, such as the presence of inflammatory cells, a thin fibrous cap (which the inflammatory cells that are present can erode sufficiently so that the plaque ruptures), and a large area containing necrotic debris and cholesterol.

What these tests also do not provide is an indication of the risk that a person without disease has of developing disease in the future. Although elevated serum cholesterol levels provide information of developing disease, cholesterol levels and other conventional risk factors are of limited predictive value. For instance, 50% of patients with CAD have normal values of traditional risk factors, including cholesterol levels. Moreover, these conventional risk factors provide limited help in predicting which patients with CAD are at risk of plaque rupture.

Accordingly, a need exists to develop novel assays for aiding in predicting who is at risk of developing atherosclerotic vascular disease, and among those who have the disease, who are at risk of experiencing plaque rupture which depending on the site of the plaque could produce myocardial infarction, stroke, critical limb ischemia, or other vascular event. The invention provides these and other assays.

### SUMMARY OF THE INVENTION

The invention broadly relates to the diagnosis of vascular disease. One aspect of the invention relates to the diagnosis of atherosclerosis, coronary artery disease and/or cardiovascular events, especially those associated with atherosclerosis. As described herein, elevated resistin levels are an indicator of vascular disease and the instability of plaques produced by the vascular disease. The invention relates, in part, to methods and reagents for diagnosing and aiding in the diagnosis of vascular conditions, assessing the likelihood of developing a vascular condition, or of developing a vascular event, and assessing the prognosis of vascular conditions and/or vascular events. The invention also provides a simple serological assay that may be used to predict, or aid in predicting, whether an individual is likely to develop a vascular event(s), which makes it possible to provide treatment or regular diagnostic testing. Thus, the invention relates to a method for predicting the likelihood that an individual will develop or will have in the future a vascular disease, a method for diagnosing or aiding in the diagnosis of a vascular disease, and a method of predicting the likelihood of having symptoms associated with a vascular disease. The present invention makes it possible to identify individuals at increased risk of developing vascular events, such as but not limited to, strokes and myocardial infarction, and provide treatment to prevent such events or reduce their severity.

### BRIEF DESCRIPTION OF THE DRAWINGS

FIGS. 1A-1B show a comparison between resistin expression in ApoE−/− and in normal mice. FIG. 1A: Changes in resistin expression in C57BL/6J wild-type and ApoE−/− mouse aorta over time, as determined by TaqMan. All results were normalized to the 3 week C57BL/6J wild-type value. The results shown are an average of four different TaqMan experiments. FIG. 1B: Immunohistochemical staining of murine aorta: Left panel: ApoE−/− aorta negative control (no primary antibody). Right panel: ApoE−/− aorta stained for murine resistin.

FIGS. 2A-2B show expression of secreted proteins by murine aortic endothelial cells in response to recombinant resistin. FIG. 2A: MCP-1 levels measured in conditioned medium after 48 hours incubation (*p=0.004). FIG. 2B: sVCAM-1 levels measured in conditioned medium after 24 hours incubation (*p=0.001).

FIGS. 3A-3C show immunohistochemical staining of human samples. FIG. 3A: Upper panel: Carotid endarterectomy sample negative control (no primary antibody). Lower panel: Carotid endarterectomy sample stained for human resistin. FIG. 3B: Left panel: Internal mammary artery (IMA) negative control (no primary antibody). Right panel: IMA stained for human resistin. FIG. 3C: Human serum levels of resistin in individuals with normal coronary arteries (open bar), and individuals with premature coronary artery disease (black bar), (*p=0.002).

FIG. 4 shows a graphical representation of the estimated probability of having premature coronary artery disease based on serum resistin levels in ng/ml for a population.

### DETAILED DESCRIPTION OF THE INVENTION

### I. Overview

The invention provides novel methods of diagnosing or aiding in the diagnosis of an existing vascular condition or in determining the risk that an individual without a vascular condition has of developing such a condition in the future, and methods of assessing if an individual is at risk of developing a vascular event. The invention is based, in part, on the unexpected findings by applicants that (a) resistin mRNA and protein levels are elevated in the aortas of atherosclerotic C57BL/6J apoE−/− mice; (b) human carotid endarterectomy samples that have atherosclerosis present stain positive for resistin protein, while internal mammary artery without evidence of atherosclerosis do not show strong staining; and (c) individuals diagnosed with premature coronary artery disease (PCAD) have higher serum levels of resistin than normal controls.

The methods and compositions described herein can be used in the diagnosis and prognosis of various forms of vascular conditions and vascular events. Moreover, the methods and compositions of the present invention can also be used to facilitate the treatment of vascular disease in individuals and the development of additional diagnostic indicators.

The present invention includes methods of predicting the risk of developing vascular disease and diagnosing vascular diseases, events, or conditions, particularly those which are associated with or mediated by expression of resistin. One aspect of the invention provides a method of diagnosing premature coronary artery disease in an individual, including previously undiagnosed individuals or individuals without disease who are at risk of developing disease. In one embodiment, the method comprises obtaining a biological sample from the individual, determining the level of resistin (e.g. amount or biological activity) in the sample and comparing it with the level of resistin in a control sample, such as but not limited to, a sample from a normal person who does not have the vascular condition or event. A higher level of resistin in the sample from the individual being assessed, compared with the level of resistin in the control sample, is an indication that the individual has, or is at high risk of developing, a vascular condition, and therefore is also at an elevated risk of developing a vascular event.

The invention further provides a method of predicting the likelihood, or aiding in predicting the likelihood, that an individual (e.g. a human) will experience a vascular condition such as atherosclerosis, coronary artery disease (CAD), a cardiovascular disease associated with atherosclerosis or CAD, or a vascular event. In one embodiment, the method comprises obtaining a biological sample from an individual to be assessed for the likelihood of developing such a condition; determining the level of a resistin gene product in the sample (i.e., the test level) and comparing the test level with a control level, wherein if the test level is greater than the level of the gene product in a control sample, the individual has an increased likelihood of developing the condition. The control can be a sample from an individual who does not have atherosclerosis or CAD. In one specific embodiment, the biological sample is a blood sample, serum sample or a plasma sample.

A related aspect of the invention is a method of diagnosing a vascular condition in an individual, the method comprising (a) determining the level of a resistin gene product in a biological sample obtained from the individual; and (b) comparing the level determined in step (a) with the level of gene product in a control level, wherein if the level determined in (a) is greater than the level of gene product in the control level, the individual is determined to have the vascular condition.

Another aspect of the invention relates to a method of diagnosing a vascular condition in an individual, the method comprising (a) determining the level of a resistin gene product in a biological sample obtained from the individual and (b) correlating the resistin level with the presence or absence of the vascular condition in the individual. In one embodiment, in the correlating step, the level of the resistin gene product in the sample is compared to a control level of resistin gene product, wherein if the level of resistin gene product is above the control level then the individual is diagnosed as having or likely having the vascular condition.

Another aspect of the invention is a method for predicting the likelihood that an individual will experience a vascular event, the method comprising (a) determining the level of a resistin gene product in a biological sample obtained from the individual, and (b) comparing the level determined in step (a) with an the level of gene product in a control, wherein if the level determined in step (a) is than the level in the control, the individual is determined to have an increased likelihood of experiencing the vascular event.

The biological sample obtained from the individual includes, but is not limited to, whole blood, blood serum and blood plasma. In another embodiment, the sample comprises peripheral blood mononuclear cells (PBMCs), such as a whole blood sample or a purified/enriched preparation of PBMCs. In another embodiment, the sample is an adipocyte biopsy.

The phrase “predicting the likelihood of developing” as used herein refers to methods by which the skilled artisan can predict onset of a vascular condition or event in an individual. The term “predicting” does not refer to the ability to predict the outcome with 100% accuracy. Instead, the skilled artisan will understand that the term “predicting” refers to forecast of an increased or a decreased probability that a certain outcome will occur; that is, that an outcome is more likely to occur in an individual exhibiting elevated resistin levels.

The methods of the present invention may be used with a variety of contexts and to assess the status of a variety of individuals. For example, the methods may be used to assess the status of individuals with no previous diagnosis of vascular disease, or with no significant cardiovascular risk factors. Cardiovascular risk factors include but are not limited to cholesterol, HDL cholesterol, systolic blood pressure, cigarette smoking, exercise, alcohol, race, family history of premature coronary artery disease, and medication use, including aspirin, statins, B-blockers and hormone replacement therapy in women. The methods are also useful to assess the status of individuals not previously known or diagnosed as having type I diabetes, type II diabetes, sugar intolerance, hyperglycemia or insulin resistance, as well as in individuals known or diagnosed as having one or more of these conditions. The individual may also be one not displaying chest pain, abnormal electrocardiograms, elevated levels of ischemic markers, necrosis markers, or thrombin/fibrin generation markers. In individuals having one or more risk factors for vascular conditions, measurement of resistin levels may provide an indication of an added risk of either developing vascular disease or of developing one of its complications.

Individuals for whom the diagnostic methods of the invention may be applied also include individuals who do not have clinically high plasma LDL-C levels or clinically low plasma HDL-C levels, or who are not being treated with PPARγ ligands. PPARγ ligands include thiazolidinediones such as ciglitazone, rosiglitazone, and pioglitazone. Other individuals how may benefit from the methods of the invention include both individuals who do not have a renal condition as well as those that do. Renal conditions include diabetic nephropathy, glomerular nephritis, end-stage renal failure, glomerular sclerosis, chronic renal failure, acute renal failure and proteinuria. Other individuals who may benefit from the methods of the invention also include those who have not been previously clinically diagnosed as having a vascular condition such as premature coronary artery disease, coronary artery disease or atherosclerosis.

II. Analysis of Resistin mRNA Levels

The amino acid sequence and the cDNA sequence of human resistin, also called FIZZ3 or Adipocyte Secreted Factor (ADSF), is disclosed in U.S. Pat. No. 6,503,730 issued Jan. 7, 2003, hereby incorporated by reference in its entirety. Human resistin sequences are also described in public sequence databases, such as Genbank Accession Nos. Q9HD89 and NP_{—}065148 (polypeptides) and NM_{—}020415 (mRNA). Splice isoforms of human resistin have also been described, including those lacking exon 2 (see Genbank Accession No. AB 111910 and Nohira T. at el. Eur J Endocrinol. 2004; 151(1):151-4).

In one embodiment of the methods described herein, determining a level of a resistin gene product in a biological sample obtained from an individual comprises determining the level of resistin mRNA in the sample. The level of resistin mRNA in the sample can be assessed by combining oligonucleotide probes derived from the nucleotide sequence of resistin with a nucleic acid sample from the individual, under conditions suitable for hybridization. Hybridization conditions can be selected such that the probes will hybridize only with the specified gene sequence. In one specific embodiment, conditions can be selected such that the probes will hybridize only with an altered nucleotide sequences, such as but not limited to, splice isoforms, and not with unaltered nucleotide sequences; that is, the probes can be designed to recognize only particular alterations in the nucleic acid sequence of resistin, including addition of one or more nucleotides, deletion of one or more nucleotides or change in one or more nucleotides, (including substitution of a nucleotide for one which is normally present in the sequence). In one specific embodiment, the oligonucleotide probe hybridizes to the resistin mRNA sequence set forth as Genbank Deposit No. NM_{—}020415, or to the coding region of the mRNA sequence.

Methods of quantitating mRNA in a sample are well-known in the art. In a particular embodiment, oligonucleotide probes specific to resistin can be displayed on an oligonucleotide array or used on a DNA chip, as described in WO 95/11995. The term “microarray” refers to an array of distinct polynucleotides or oligonucleotides synthesized on a substrate, such as paper, nylon or other type of membrane, filter, chip, glass slide, or any other suitable solid support. Microarrays also includes protein microarrays, such as protein microarrays spotted with antibodies. Other techniques for detecting resistin mRNA levels in a sample include reverse transcription of mRNA, followed by PCR amplification with primers specific for a resistin mRNA.

### III. Analysis of Resistin Polypeptide Levels

In one embodiment of the methods described herein, determining a level of a resistin gene product in a biological sample obtained from the individual comprises determining the level of resistin polypeptide in the sample. Human resistin comprises various conserved domains including, but not limited to, a putative signal peptide from about amino acid residue 1 to about amino acid residue 18, and a putative secreted portion comprising from about amino acid residue 19 to about amino acid residue 108. The secreted portion is characterized by conserved cysteine residues at amino acid positions 22, 51, 63, 72, 74, 78, 89, 91, 93, 103, and 104, in human resistin (See, for example, SEQ ID NO:4 in U.S. patent Publication No. 2002/0161210). A crystal structure of human resistin has been described by Patel et al. (2004) Science; 304(5674):1154-8. The crystal structure consists of coiled-coil trimers that form tail-to-tail hexamers through disulfide bonds near their amino termini. According to Patel et al., the trimers are further interlinked to form tail-tail hexamers. To achieve this linkage, the amino-terminal coiled-coil tips from each of the two trimers splay apart to enable tail-to-tail interdigitation, forming a short antiparallel six-helix bundle. Each helix from a trimer coiled coil is disulfide bonded through Cys6 to a Cys6 from the opposing trimer. Raghu et al (2004) Biochem Biophys Res Commun. 313(3):642-6 also describes the formation of disulfide linked dimers between resistin polypeptides mediated by cysteine 22 in human resistin.

In one embodiment of the methods described herein, determining a level of a resistin gene product comprises determining the level of a putative secreted portion of human resistin in the sample, such as but not limited to, a secreted resistin polypeptide spanning from amino acid residue 19 to 108.

In another specific embodiment, determining a level of a resistin gene product in the sample comprises determining the level of resistin trimers in the sample. In yet another specific embodiment, determining a level of a resistin gene product in the sample comprises determining the level of disulfide-linked resistin dimers (the dimers formed by the disulfide bond formation between one resistin polypeptide in one trimer and another resistin polypeptide in another trimer). In another specific embodiment, determining a level of a resistin gene product in the sample comprises determining the level of resistin polypeptide hexamers in the sample. In yet another specific embodiment, determining a level of a resistin gene product in the sample comprises determining the level of resistin polypeptide monomers, dimers, trimers or hexamers, or combinations thereof, or determining a ratio of two or more multimeric forms, such as the ratio or trimers to hexamers. In yet another specific embodiment, determining a level of a resistin gene product comprises determining the total level of secreted resistin polypeptides in the sample, regardless of their oligomeric state. Resistin monomers, disulfide-linked dimers, trimers and hexamers (disulfide-linked trimers) may detected and distinguished from each other by using combinations of gel filtration chromatography, reducing/nonreducing SDS-PAGE, and western blotting, as described in Patel et al. and/or Raghu et al. cited above.

The level of a resistin polypeptide can be determined by contacting the biological sample with an antibody which specifically binds to resistin and determining the amount of bound antibody, e.g., by detecting or measuring the formation of the complex between the antibody and a resistin polypeptide. Antibodies may be used which bind to a secreted form of resistin, or to altered forms of the resistin protein, including addition proteolytic products. In one embodiment, the antibodies bind to an N-terminally truncated resistin polypeptide lacking cysteine 22. Antibodies can be monoclonal, polyclonal or a mixture thereof.

The term antibody as used herein is intended to include whole antibodies, e.g., of any isotype (IgG, IgA, IgM, IgE, etc) and fragments which are also specifically reactive with resistin or a complex comprising resistin. Antibodies can be fragmented using conventional techniques and the fragments screened in the same manner as described above for whole antibodies. For example, F(ab′)₂ fragments can be generated by treating antibody with pepsin. The resulting F(ab′)₂ fragment can be treated to reduce disulfide bridges to produce Fab′ fragments. The antibodies used in the present invention is further intended to include bispecific and chimeric molecules, as well as single chain (scFv) antibodies.

The resistin antibodies may include trimeric antibodies and humanized antibodies, which can be prepared as described, e.g., in U.S. Pat. No: 5,585,089. Single chain antibodies may also be used to detect levels of resistin polypeptides. All of these modified forms of antibodies as well as fragments of antibodies are intended to be included in the term “antibody”. Antibodies which bind to resistin may also be obtained commercially. For example, a purified IgG Antibody specific for residues 51-108 of human resistin may be purchased from Phoenix Pharmaceuticals, Inc., 530 Harbor Boulevard, Belmont, Calif. 94002, U.S.A. Alternatively, a rabbit polyclonal antibody to human resistin may also be purchased from BioVision, Inc, 980 Linda Vista Avenue, Mountain View, Calif. 94043.

The antibodies can be labeled (e.g., radioactive, fluorescently, biotinylated or HRP-conjugated) to facilitate detection of the complex. Appropriate assay systems for detecting resistin polypeptide levels include, but are not limited to, Enzyme-Linked Immunosorbent Assay (ELISA), competition ELISA assays, Radioimmuno-Assays (RIA), immunofluorescence, western, and immunohistochemical assays which involve assaying a resistin gene product in a sample using antibodies having specificity for resistin. Numerous methods and devices are well known to the skilled artisan for the detection and analysis of resistin of the instant invention. With regard to polypeptides or proteins in test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. These devices and methods can utilize labeled molecules in various sandwich, competitive, or non-competitive assay formats, to generate a signal that is related to the presence or amount of an analyte of interest. Additionally, certain methods and devices, such as but not limited to, biosensors and optical immunoassays, may be employed to determine the presence or amount of analytes without the need for a labeled molecule. See, e.g., U.S. Pat. Nos. 5,631,171 and 5,955,377, each of which is hereby incorporated by reference in its entirety, including all tables, figures and claims.

An amplified immunoassay, such as but not limited to, immuno-PCR can also be used. In this technique, the antibody is covalently linked to a molecule of arbitrary DNA comprising PCR primers, whereby the DNA with the antibody attached to it is amplified by the polymerase chain reaction. See E. R. Hendrickson et al., Nucleic Acids Research 1995; 23, S22-529 (1995) or T. Sano et al., in “Molecular Biology and Biotechnology” ed. Robert A. Meyers, VCH Publishers, Inc. (1995), pages 458-460.

Levels of resistin polypeptides may also be determined using protein microarrays. Methods of producing protein microarrays that may be adapted for detecting levels of resistin protein in a clinical sample are described in the art (see for example of Xiao et al. (2005) Mol Cell Endocrinol.; 230(1-2):95-10; Protein Microarrays (2004) Mark Schena (Ed) Jones & Bartlett Publishers, Inc.). U.S. Patent Pub. 2003/0153013 describes methods of detecting proteins, e.g. antigens or antibodies, by immobilizing antibodies in a protein microarray on a membrane and contacting the microarray with detection proteins which can bind to the proteins to form protein complexes. Similarly, U.S. Patent Pub. 2004/0038428 describes methods of constructing protein microarrays.

Alternatively, the level of resistin polypeptide may be detected using mass spectrometric analysis. Mass spectrometric analysis has been used for the detection of proteins in serum samples (see for example Wright et al. (1999) Prostate Cancer Prostatic Dis 2:264-76, and Petricoin et al. (2002) Lancet.; 359 (9306): 572-7). U.S. Patent No. 2003/0013120 describes a system and method for differential protein expression and a diagnostic biomarker discovery system that may be adapted for measuring levels of resistin polypeptides in a fluid sample. Mass spectroscopy methods include Surface Enhanced Laser Desorption Ionization (SELDI) mass spectrometry (MS), SELDI time-of-flight mass spectrometry (TOF-MS), Maldi Qq TOF, MS/MS, TOF-TOF, ESI-Q-TOF and ION-TRAP.

In one embodiment of the methods described herein, determining the level of a resistin gene product in a biological sample comprises determining the level of a resistin polypeptide having a post-translational modification, such as a phosphorylated, glycosylated or proteolytic processed resistin polypeptide. Phosphorylation can include phosphorylation of a tyrosine, serine, threonine or histidine. Antibodies that can be used to detect these modifications can include phosphotyrosine-specific antibody, phosphoserine-specific antibody, phosphoserine-specific antibody, and phosphothreonine-proline antibody, for example. Proteolytic processing may be detected by using antibodies specific for a cleaved product or by amino acid sequencing of the resistin protein.

### IV. Analysis of Resistin Bioactivity

In one embodiment of the methods described herein, determining the level of a resistin gene product in the biological sample comprises determining the level of resistin bioactivity i.e. (a biological activity of a resistin polypeptide). The term “resistin bioactivity” refers to the biochemical and physiological roles played by a resistin polypeptide in a cell or in an organism. It includes the ability of resistin to effect a molecular change, such as but not limited to, a change in gene expression in a cell contacted with a resistin polypeptide. For example, Verma et al. (2003) Circulation.;108(6):736-40, describes a bioassay for determining bioactivity of resistin by contacting human saphenous vein endothelial cells (HSVEC) with resistin and measuring induction of transcription from the ET-1 promoter. Calabro et al. (2004) Circulation.;110(21): 3335-40 teach a bioassay where resistin polypeptide promotes the division of human aortic smooth muscle cells (HASMCs). Muse et al. (2004) J. Clin Invest.; 114(2): 232-9 describes the reconstitution of hepatic insulin resistance in mice that were both fed a high fat diet and treated with an antisense oligonucleotide directed to resistin. The Examples provided in the exemplification include additional assays for determining the bioactivity of resistin polypeptides.

### V. Control Samples

In one embodiment of the methods described herein, the control level is the level of a resistin gene product in a sample from a normal individual, such as but not limited to, an individual who does not have the vascular condition. In a specific embodiment, the normal individual is one not having atherosclerosis, PCAD or CAD. If the control sample is from a normal individual, then increased levels of the resistin gene product in the biological sample from the individual being assessed compared to the control level indicates that the individual has an increased risk of developing the vascular condition or of experiencing the vascular event.

The control level of resistin gene product can be determined at the same time as the level of resistin gene product in the biological sample from the individual. Alternatively, the control level may be a predetermined standard value, or range of values, (e.g. from analysis of other samples) to correlate with increased risk of having the vascular condition or of developing the vascular event. In one specific embodiment, the control value may be data obtained from a data bank corresponding to currently accepted normal levels the resistin gene product under analysis. In situations, such as but not limited to, those where standard data is not available, the methods of the invention may further comprise conducting corresponding analyses in a second set of one or more biological samples from individuals not having the vascular condition, in order to generate the control level. Such additional biological samples can be obtained, for example, from unaffected members of the public.

In one embodiment, the control level has been normalized according to at least one parameter, such as but not limited to, age, sex, body mass index (BMI) or cardiovascular risk factors, to increase the predictive accuracy. In another embodiment, the control level is normalized to another marker level in the individual, such as but not limited to, fasting sugar level, LDL, HDL, levels, triglycerides or total cholesterol in blood plasma. Other methods of normalizing are with reference to a disorder, condition or risk factors. For example, there could be a set of control values for diabetics and one for nondiabetics.

One skilled in the art would appreciate that the control level of resistin gene product may vary according to the resistin gene products (e.g. mRNA, monomer, multimers, posttranslationally-modified forms) that are detected, the type of biological sample, the handling of the biological sample and/or the method used to detect the gene product. For example, prolonged storage of a serum sample, or repeated freeze-thaw cycles, may result in degradation and loss of detectable signal from a resistin gene product. Likewise, the control level may vary according to the affinity of an antibody for a resistin polypeptide. In one embodiment, the control level is a control level that has been determined using the same type of biological sample, comparable handling of the biological sample, same type of resistin gene product and same detection technique as for the individual being tested.

In one specific embodiment, the control level of resistin in a serum sample using an ELISA antibody assay, such as the Bio Vendor resistin ELISA kit, is at least about 12 ng/ml, 14.5 ng/ml, or 16.5 ng/ml. In another embodiment of the methods described herein, the sample is a serum or plasma sample, the resistin gene product is a resistin polypeptide, and the individual is said to have an increased likelihood of developing the vascular condition of about from 1.06 to about 1.09 for every increase of from about 0.8 to 1.2 ng/ml of resistin polypeptide over the control level, whereas in a related embodiment the individual is said to have an increased likelihood of developing the vascular condition of about 1.075 for every increase of about 1 ng/ml of resistin polypeptide in serum over the control level.

In the methods of the invention, the comparison of the resistin gene product level with the control level can be a straight-forward comparison, such as but not limited to, a ratio. The comparison can also involve subjecting the measurement data to any appropriate statistical analysis. In the diagnostic procedures of the invention, one or more biological samples obtained from an individual can be subjected to a battery of analyses in which a desired number of additional genes, gene products, metabolites, and metabolic by-products are measured. In any such diagnostic procedure it is possible that one or more of the measures obtained will produce an inconclusive result. Accordingly, data obtained from a battery of measures can be used to provide for a more conclusive diagnosis and can aid in selection of a normalized control level of resistin expression. It is for this reason that an interpretation of the data based on an appropriate weighting scheme and/or statistical analysis may be desirable in some embodiments.

In another embodiment, abnormal resistin levels are combined with the results of assessment of other risk factors to determine cumulative risk. For example, an individual with an elevated resistin level and an elevated cholesterol level would be at greater risk of having or of developing atherosclerotic vascular disease than an individual for whom only one of these values is abnormal. Resistin levels can be combined with other risk factors such as elevated homocysteine or CRP serum levels. Further, resistin levels can be combined with one or more other risk factors, where the greater the number of risk factors and the greater the level of resistin, the greater the likelihood of having the vascular condition.

### VI. Vascular Conditions/Events

The vascular conditions in the methods described herein include diseases or disorders of a blood vessel and/or relating to the circulation of, for example, the heart (e.g., cardiovascular condition) or brain (e.g., cerebrovascular condition). Vascular conditions which can be assessed/predicted using the methods described herein include, but are not limited to, atherosclerosis, premature coronary artery disease (PCAD), coronary artery disease (CAD), cerebral atherosclerosis or peripheral vascular disease (PVD). Vascular conditions also include disorders that gradually lead to chronic or acute ischemia, including the stage of the disorder at which such ischemia is not yet evident. As used herein, the term “vascular conditions” does not encompass type I diabetes, type II diabetes, sugar intolerance, insulin resistance or hyperglycemia.

“Coronary artery disease” (“CAD”) is a pathological state characterized by a diseased blood vessel wall having among other constituents, inflammatory cells, cholesterol, necrotic debris, fibrotic tissue, and excessive smooth muscle cells. If the vascular disease results in narrowing of the artery of about 50% or more, it can lead to insufficient oxygen delivery to cardiac muscle, legs, or the brain, in which case the condition may be associated with some dysfunction of the supplied tissue.

In some embodiments, the vascular condition is a vascular event. Vascular events refer to acute conditions of the vascular system. As used in this disclosure, vascular events include disorders in which symptomatic and/or asymptomatic ischemia of the supplied tissue occurs (e.g., angina pectoris and myocardial infarction, claudication, or stroke). Vascular events include cardiovascular and/or cerebrovascular events, such as but not limited to, thrombotic occlusion of the vessel lumen. A thrombotic disorder/event occurs, for example, when a clot forms and lodges within a blood vessel. The blockage may fully block or partially block the blood vessel, causing tissue ischemia. Two phases of a cardiovascular and/or cerebrovascular event can exist, an ischemic stage and a necrotic stage. An individual may suffer from ischemia in which a decrease of blood flow may occur. This decrease in blood flow causes a decrease in tissue oxygenation. After prolonged ischemia, the tissue may undergo necrosis (death of the tissue).

Other examples of cardiovascular and/or cerebrovascular events include myocardial infarction (MI), cardiac arrest, angina, unstable angina, stroke, transient ischemic attack (TIA), coronary death, non-fatal myocardial infarction, deep venous thrombosis, pulmonary embolism, critical limb ischemia, thrombotic re-occlusion subsequent to a coronary intervention procedure

The term “cardiovascular disease/events associated with atherosclerosis” encompasses diseases that are medically linked to atherosclerosis in that they are a consequence of atherosclerotic lesions. Cardiovascular diseases associated with atherosclerosis that may be mentioned include coronary artery disease, myocardial infarction, peripheral vascular disease including claudication and critical limb ischemia, strokes and TIAs.

### VII. Additional Diagnostic/Predictive Markers

In certain embodiments, assessment of one or more additional markers are combined to increase the predictive value of the analysis in comparison to that obtained from measurement of the resistin gene product alone. For example, one or more markers for myocardial injury, coagulation, or atherosclerotic plaque rupture can be measured along with resistin, to enhance the predictive value of the described methods.

In one embodiment, assessment of one or more additional markers indicative of atherosclerotic plaque rupture is combined with resistin. An atherosclerotic plaque consists of inflammatory cells, accumulated lipids, smooth muscle cells, connective tissue, and glycosaminoglycans. Vessels containing such plaques have reduced systolic expansion, abnormally rapid wave propagation, and progressively reduced elasticity as plaque formation progresses. A plaque may progress to severe stenosis and total arterial occlusion, almost invariably because of acute rapture of the plaque with resulting occlusion of the vessel (either partial or complete) by thrombus. Some plaques are stable, but others, referred to as unstable plaques, are rich in lipids and inflammatory cells, typically have a thin fibrous cap and may undergo spontaneous rupture. These unstable plaques are closely associated with the onset of an acute ischemic event. Therefore, markers of atherosclerotic plaque rupture may be useful in the diagnosis and vascular conditions. Such markers of atherosclerotic plaque rupture include human neutrophil elastase, inducible nitric oxide synthase, lysophosphatidic acid, malondialdehyde-modified low-density lipoprotein, matrix metalloproteinase-1, matrix metalloproteinase-2, matrix metalloproteinase-3, and matrix metalloproteinase-9.

In one embodiment, assessment of one or more additional markers indicative of coagulation is combined with resistin. The coagulation cascade is an enzymatic pathway that involves numerous serine proteinases normally present in an inactive, or zymogen, form. The presence of a foreign surface in the vasculature or vascular injury results in the activation of the intrinsic and extrinsic coagulation pathways, respectively. A final common pathway is then followed, which results in the generation of fibrin by the serine proteinase thrombin and, ultimately, a crosslinked fibrin clot. In the coagulation cascade, one active enzyme is formed initially, which can activate other enzymes that activate others, and this process, if left unregulated, can continue until all coagulation enzymes are activated. Coagulation markers include β-thromboglobulin, D-dimer, fibrinopeptide A, platelet-derived growth factor, plasmin-α-2-anti-plasmin complex, platelet factor 4, prothrombin fragment 1+2, P-selectin, thrombin-antithrombin III complex, thrombus precursor protein, tissue factor and von Willebrand factor.

In another embodiment, additional markers assessed can be, for example, soluble tumor necrosis factor-α receptor-2, interleukin-6, and lipoprotein-associated phospholipase A2, C-reactive protein (CRP), Creatine Kinase with Muscle and/or Brain subunits (CKMB), thrombin anti-thrombin (TAT), soluble fibrin monomer (SFM), fibrin peptide A (FPA), myoglobin, thrombin precursor protein (TPP), platelet monocyte aggregate (PMA) troponin and homocysteine. In another embodiment, the additional markers can be Annexin V, B-type natriuretic peptide (BNP) which is also called brain-type natriuretic peptide, enolase, Troponin I (TnI), cardiac-troponin T, Creatine kinase (CK), Glycogen phosphorylase (GP), Heart-type fatty acid binding protein (H-FABP), Phosphoglyceric acid mutase (PGAM) and S-100. In a further embodiment, the additional marker is C-reactive protein (CRP). C-reactive protein is a (CRP) is a homopentameric Ca²⁺-binding acute phase protein with 21 kDa subunits that is involved in host defense. CRP preferentially binds to phosphorylcholine, a common constituent of microbial membranes. In another embodiment, the one or more additional marker is Annexin V which is also called lipocortin V, endonexin II, calphobindin I, calcium binding protein 33, placental anticoagulant protein I, thromboplastin inhibitor, vascular anticoagulant-α, or anchorin CII.

In another embodiment, the one or more blood-derived additional markers are anti-heat shock protein 60 (HSP60) antibodies, heat shock protein 70 (HSP70), and myeloperoxidase (MPO). Anti-HSP60 antibodies may be detected using a solid-phase immobilized HSP60 polypeptide or HSP60 peptide fragments and a tagged antibody capable of recognizing the non-variable region of the anti-hsp60 antibody to be detected, such as tagged anti-human Fab. HSP60 peptide fragments that may be used to detect the presence of anti-HSP60 antibodies in a sample are described in U.S. Pat. No. 6,110,746. U.S. Patent Publication No. 2005/0042678 to Epstein et al. describes assays for detecting anti-HSP60 antibodies and HSP70 in biological samples. The diagnostic methods described in Epstein et al. may be combined with the methods of the present invention to increase the predictive accuracy of the resistin-based screening methods.

In another embodiment, the additional marker(s) is a polypeptide, such as adiponectin, leptin or adrenomedullin. Antibodies to adrenomedullin are described in U.S. Pat. No. 5,837,823. Adiponectin (also called ACRP30, adipoQ or GBP28) is a protein secreted from adipocytes.

In embodiments where one or more markers are used in combination with resistin to increase the predictive value of the analysis, the level of the additional marker(s) may be measured in the same biological sample from the individual or in another, which may be of the same type or of a different type. For example, the level of resistin polypeptide may be measured in a sample of blood plasma, while the level of an additional marker, such as but not limited to, CRP or homocysteine, may be measured in the same sample of plasma, in another sample of plasma, or in a sample of serum from the individual.

### EXEMPLIFICATION

The invention now being generally described, it will be more readily understood by reference to the following examples, which are included merely for purposes of illustration of certain aspects and embodiments of the present invention and are not intended to be limiting in any way.

The contents of any patents, patent applications, patent publications, or scientific articles referenced anywhere in this application are herein incorporated by reference in their entirety.

### Summary and Introduction to of Experimental Results

Resistin, an adipocyte-derived cytokine linked to insulin resistance and obesity, can activate endothelial cells (ECs). Using microarrays, applicants found that along with numerous other pro-atherosclerotic genes, resistin expression levels are elevated in the aortas of C57BL/6J apoE−/− mice; these findings led us to further explore the relation between resistin and atherosclerosis. Using TaqMan PCR and immunohistochemistry, applicants found that ApoE−/− mice had significantly higher resistin mRNA and protein levels in their aortas, and elevated serum resistin levels, compared to C57BL/6J wild-type mice. Incubation of murine aortic ECs with recombinant resistin increased monocyte chemoattractant protein (MCP)-1 and soluble vascular cell adhesion molecule (sVCAM)-1 protein levels in the conditioned medium. Furthermore, human carotid endarterectomy samples stained positive for resistin protein, while internal mammary artery did not show strong staining. Individuals diagnosed with premature coronary artery disease (PCAD) were found to have higher serum levels of resistin than normal controls. In summary, resistin protein is present in both murine and human atherosclerotic lesions, and mRNA levels progressively increase in the aortas of mice developing atherosclerosis. Resistin induces increases in MCP-1 and sVCAM-1 expression in murine vascular endothelial cells, suggesting a possible mechanism by which resistin might contribute to atherogenesis. Finally, PCAD individuals exhibited increased serum levels of resistin when compared to controls. These findings suggest a possible role of resistin in cardiovascular disease.

### Methods Used in Experiments

### Animals

C57BL/6J and C57BL6/J ApoE−/− mice were bred in-house (breeders obtained from the Jackson Laboratory, Bar Harbor, Me.). All animals were housed in microisolator cages, and given free access to sterile normal chow and water. Mice were sacrificed at 3, 6, and 16 weeks of age, and blood, hearts, and aortas were collected. For the gene array study, males and females were used in equal numbers (n=20 for 3 week time point, n=16 for 6 and 16 week time points). For the PCR and serum analysis, C57BL/6J and C57BL/6J apoE−/− mice were used (n=10 for 3 week time point, n=6 for 6 and 16 week time points). This protocol was approved by the Institutional Animal Care and Use Committee at MedStar Research Institute.

### RNA Isolation and Affymetrix GeneChip Analysis

Aortas were pooled, and total RNA was extracted using TRIzol® reagent (Invitrogen) as previously described in Chen Y W et al. (2000) J Cell Biol.;151:1321-1336. The pooled samples from each group were divided and two in vitro transcription reactions were run for each group. The duplicate cRNA samples were hybridized to separate Affymetrix murine U74A ver. 2 GeneChips as previously described in Burnett M S et al. (2004) Circulation;109:893-897. Scanned raw data were processed with Affymetrix GeneChip v 5.0 software and then imported into GeneSpring 5.1 software (Silicon Genetics, Redwood City, Calif.) for further analysis, as previously described in Burnett M S et al. (2004) Circulation; 109:893-897. All data were normalized to the earliest timepoint (3 weeks of age).

### TaqMan Real Time PCR

Commercially available primer and probe sets for resistin and metalloelastase (mmp-12) were obtained from Applied Biosystems, Inc. The following primers and probe were used for confirmation of the pro-atherosclerotic gene osteopontin: Forward: 5′AGTGATTTGCTTTTGCCTGTTTGG3′; Reverse: 5′AGGCTGTAAAGCTTCTCCTCTGA3′; Probe: 5′CCCTCCCGGTGAAAGT3′. Probes were fluorescently labeled with TAMARA and FAM. TaqMan Real Time PCR was performed as previously described in Burnett M S et al. (2004) Circulation;109:893-897.

### Immunohistochemistry

All human samples were collected with consent, under a protocol approved by the Institutional Review Board at the Washington Hospital Center. Fresh frozen sections (8 μm thick) were fixed with methanol, treated with H₂O₂, and blocked with 5% goat serum in TBS, then incubated with anti-resistin antibody. (rabbit anti-mouse or anti-human resistin, Chemicon, Inc.). After washing, samples were incubated with HRP-labeled goat anti-rabbit IgG (mouse and human adsorbed) (Santa Cruz Inc.). Samples were developed using DAB (Vector Labs), and stained with hematoxylin.

Cell Culture [symbol] cells (ECs) were obtained from C57B1/6J mouse aorta and cultured as [symbol] [17]. Four to six passages were used for all experiments. ECs (10⁶ cells/mL) were incubated with medium containing 5, 50, or 100 ng/mL of resistin (US Biologicals, Inc.), for 24 or 48 hours. Recombinant resistin contained <0.1 ng/mL of endotoxin. Following the incubation, the ECs supernatant were tested immediately for MCP-1 and sVCAM-1.

### ELISA

Commercially available ELISA kits were used to measure levels of MCP-1 (BioSource, Inc.), and sVCAM-1 (R & D Systems, Inc.) in the conditioned medium, according to the manufacturers' directions. Commercially available ELISAs kits were used to measure resistin levels in mouse serum and human plasma (BioVendor, Inc.) according to the manufacturers directions.

### Biological Samples

Under a protocol approved by the Institutional Review Board at the Washington Hospital Center, blood was collected from consenting individuals undergoing coronary angiography at the Washington Hospital Center between December 2001 and July 2003. Premature CAD individuals were defined as individuals who were diagnosed with CAD by angiography or had suffered a myocardial infarction prior to age 45 (n=39). Individuals over 45 years of age with no evidence of stenosis, as determined by angiography, served as controls (n=38). There were no significant differences in traditional risk factors (sex, BMI, Glucose (non-fasting), HDL, LDL, triglycerides, and total cholesterol) between the normal and PCAD groups, with the exception of age, which was a function of the experimental design. That is, normal individuals were not selected unless they were over the age of 45 years and found (by angiography) to have completely normal coronary arteries, while PCAD individuals were younger than 45 years at the time of diagnosis (either by angiography or documented MI). Serum was isolated and stored at −80° C. until testing.

### Statistics

Data are given as mean±SEM. P values were determined using student t tests. Continuous variables are presented as the mean and one standard deviation (SD) and compared using Student's t-test. Discrete variables are presented as percentages and relative frequencies, and compared using chi-square statistics or Fisher's exact test. Logistic regression analysis was performed to evaluate the relationship between premature CAD and resistin levels.

### EXAMPLE 1

### Resistin Expression in ApoE−/− Aortas

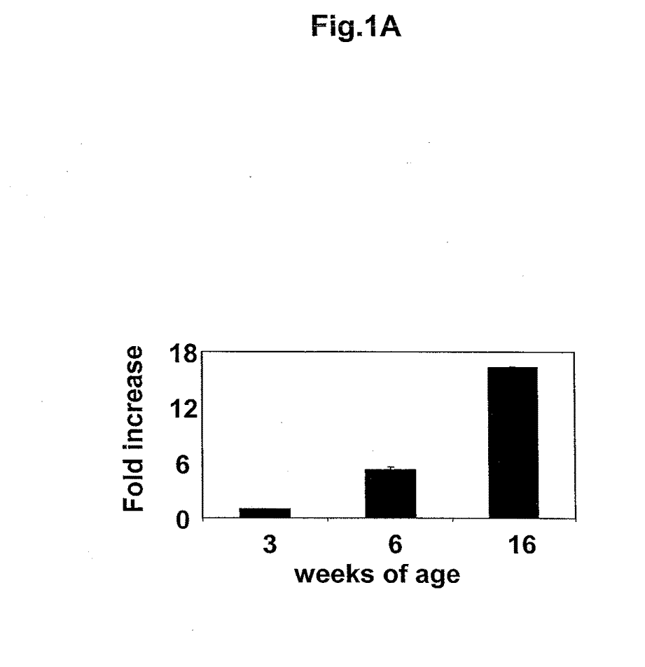

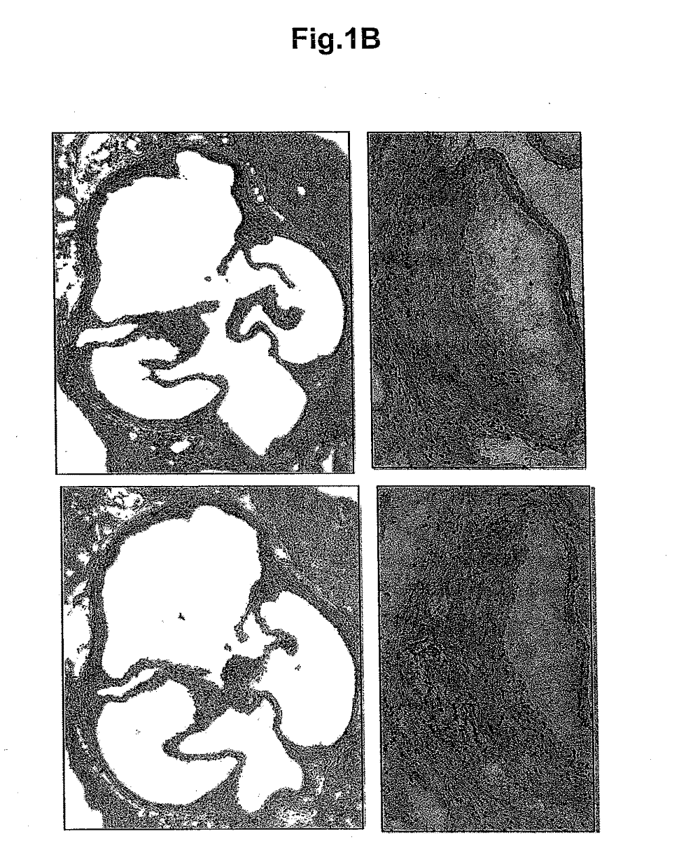

In the initial micro array analysis, many genes previously determined to be involved in atherosclerosis were found to be upregulated over time in the apoE−/− aortas. A representative group of these pro-atherosclerotic genes can be found in Table 1. Complete lists of upregulated and downregulated genes are included in Data Supplements 1 and 2, respectively. Surprisingly, applicants found that resistin mRNA levels increased over time in the ApoE−/− aortas. Differences in expression levels of resistin, osteopontin, and mmp-12 were confirmed using TaqMan (Table 1). A second group of animals were then used to compare resistin mRNA levels in both ApoE−/− and wild-type C57BL/6J− mice. While murine resistin mRNA levels increased over time in the aortas of both ApoE−/− and wild-type mice, resistin mRNA levels were significantly higher in the ApoE−/− mice at each time point. FIG. 1A is a graphical representation of changes in resistin mRNA levels, over time.

To further validate the findings from our microarray study, applicants stained aortic root samples from both apoE−/− (FIG. 1B) and wildtype mice for murine resistin. The wild-type animals, with no evidence of atherosclerotic lesions, did not show appreciable resistin staining in the aorta. The ApoE−/− mice, however, showed significant staining for murine resistin within the atherosclerotic lesions.

Serum samples from both C57BL/6J apoE−/− and C57BL/6J wild type mice were tested for murine resistin using an ELISA. At sixteen weeks of age, the atherosclerotic C57BL/6J apoE−/− mice had significantly higher serum levels of resistin, compared to the C57BL/6J control animals (p<0.001).

### EXAMPLE 2

### Resistin Induction of Gene Expression in Murine Aortic ECs

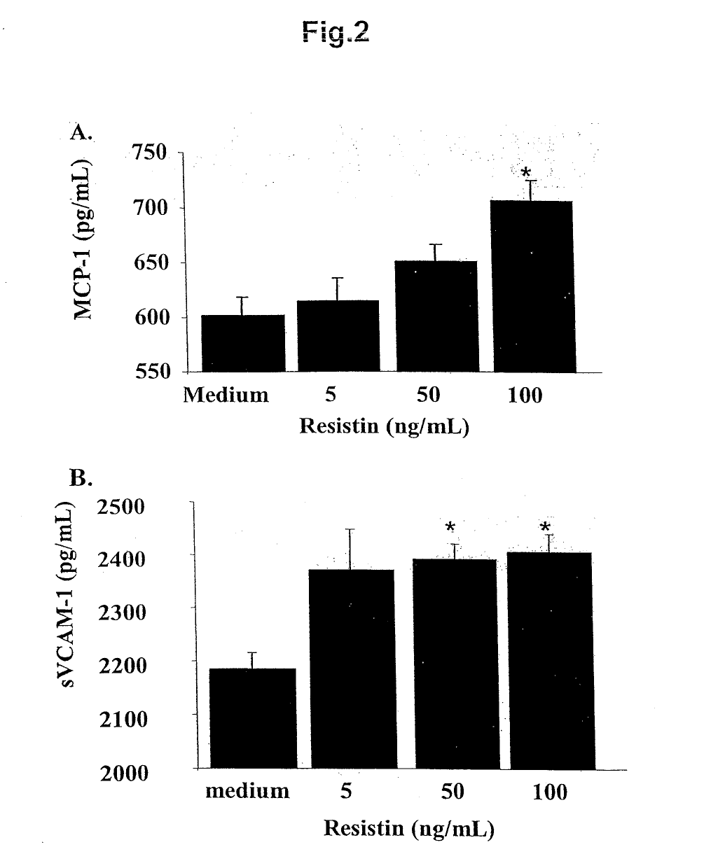

When incubated with murine aortic ECs for 24-48 hours, recombinant murine resistin increased levels of both MCP-1 and sVCAM-1 in the conditioned medium. These results are illustrated in FIG. 2.

### EXAMPLE 3

### Immunohistochemistry of Human Samples

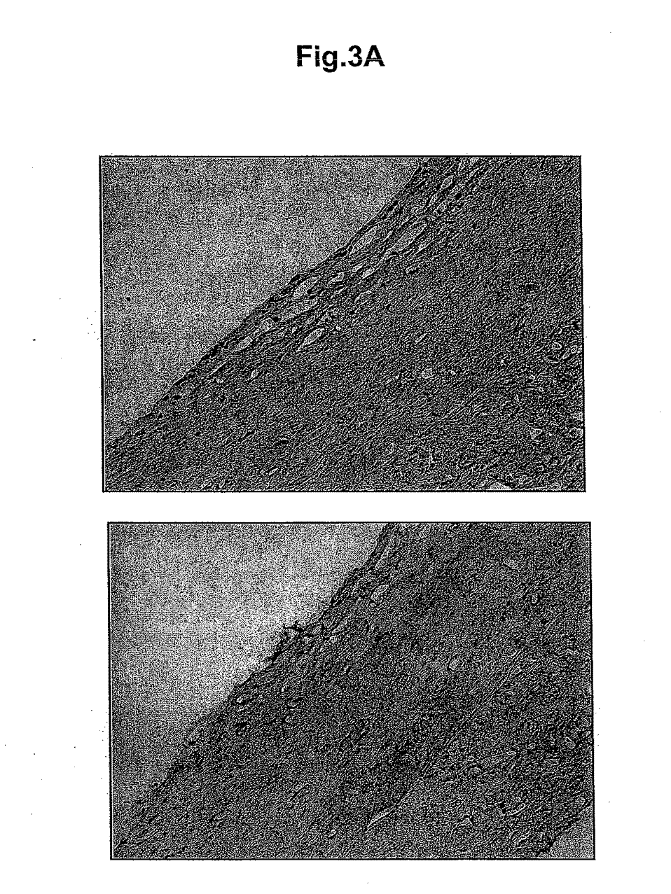

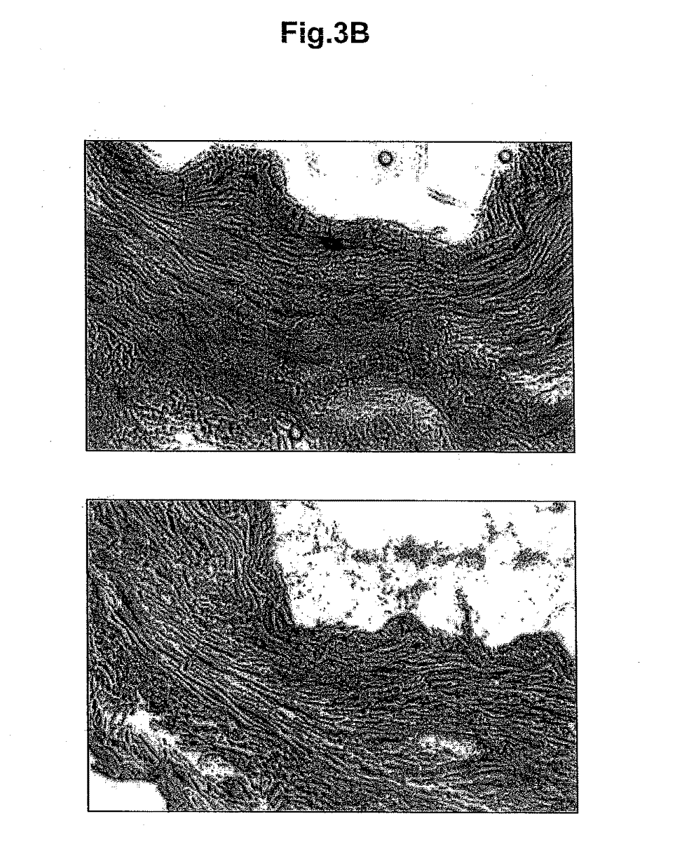

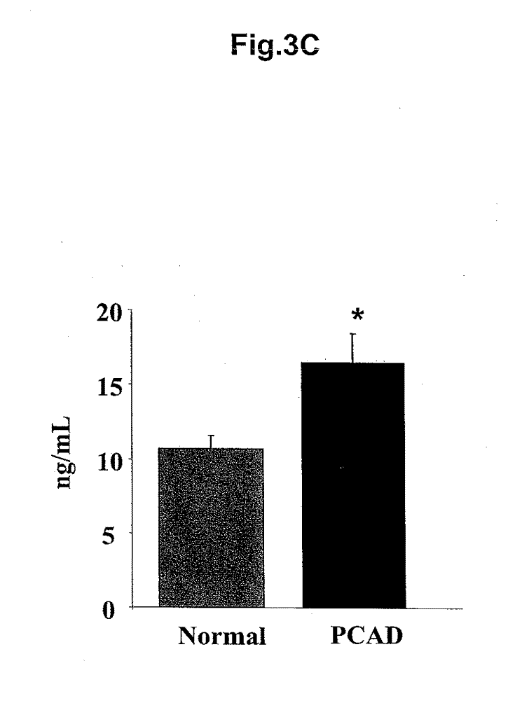

Human carotid endarterectomy samples were stained with anti-human resistin antibody; resistin was found to be present in the lesions (a representative sample is shown in FIG. 3A). An internal mammary artery (IMA) sample, with no signs of atherosclerosis, was stained to determine if resistin was present in normal arteries. While there was slight staining in the IMA, most of the immunopositivity was in the adventitia rather than in the media or intima (FIG. 3B).

### EXAMPLE 4

### Serum Resistin Levels are Elevated in Individuals having a Vascular Condition

The following risk factors were compared between the normal and premature (P)CAD individuals making up this population: sex, age, BMI, Glucose (non-fasting), HDL, LDL, triglycerides, total cholesterol, and plasma resistin levels. The results are shown in Table 2. There were no significant differences in these traditional risk factors between the normal and PCAD groups with the exception of age which, as noted above, was a function of the experimental design. In this dataset individuals with premature CAD had significantly higher plasma levels of resistin, as determined by ELISA (16.5±1.9 vs. 10.7±0.9 ng/mL, p=0.009) and shown in FIG. 3C.

This population was 69% men, and 25% diabetic. The average age of the individuals was 51.0+/−10.1 years. Men and women had similar levels of resistin (13.41 ng/ml vs. 13.92 ng/ml respectively). Diabetics trended towards higher levels of resistin, but this difference is not statistically significant (15.51 ng/ml vs. 13.09 ng/ml, p=0.361).

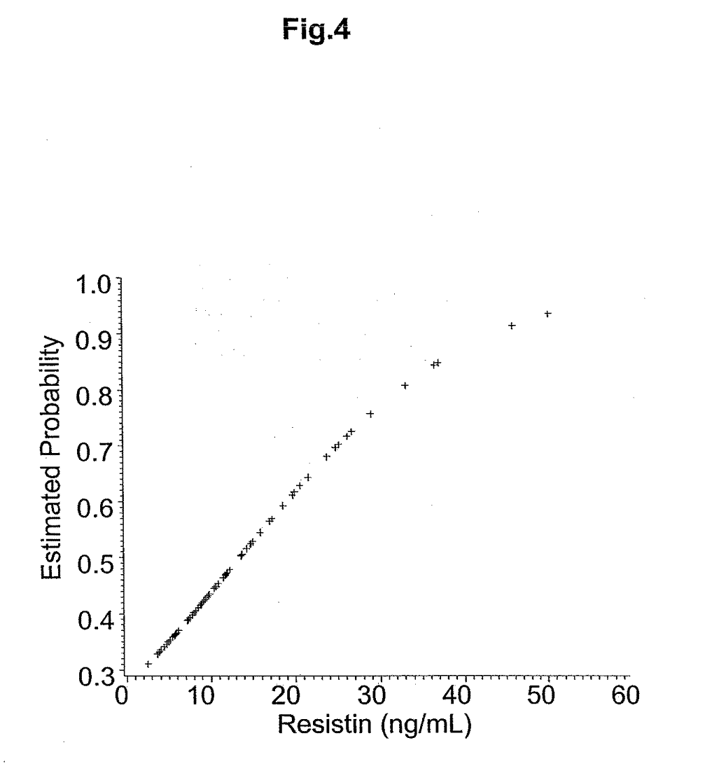

Logistic regression analysis (Table 3) revealed that individuals with higher levels of resistin (ng/ml) are more likely to have premature coronary artery disease (p=0.016; OR 1.075, CI 1.014-1.141). This finding represents an increased risk of premature CAD of 1.8 (80%) for every increase of 10 ng/ml unit of resistin. A graph depicting the estimated risk of PCAD in relation to resistin levels is shown in FIG. 4. There were no significant correlations between resistin levels and HDL (r=−0.113, p=0.39), LDL (r=0.081, p=0.58), total cholesterol (r=0.018, p=0.89), triglycerides (r=0.012, p=0.93), or BMI (r=0.019, p=0.88). Data are shown in Table 4.

TABLE 1
Gene expression changes for selected genes
during atherosclerotic development. Expression
is normalized to the three week time point.

| | Genbank Deposit | Affymetrix expression levels* | | |
|---|---|---|---|---|
| Gene Name | Number | 3 weeks | 6 weeks | 16 weeks |
| CCR2 | U56819 | 1.00 | 1.14 | 2.02 |
| Cyr61 | M32490 | 1.00 | 1.66 | 3.28 |
| Gro1 | J04596 | — | — | 4.39 |
| SAA | X03505 | — | — | 3.71 |
| VCAM1 | U12884 | 1.00 | 1.15 | 2.83 |
| PAI-1 | M33960 | 1.00 | 1.76 | 4.90 |
| HSP70-3 | M12571 | 1.00 | 2.29 | 7.51 |
| IL1r1 | M20658 | 1.00 | 1.20 | 2.29 |
| PDGFrb | X04367 | 1.00 | 2.09 | 3.38 |
| Retn | AA718169 | 1.00 | 5.26 | 16.3 (26.6)** |
| MMP-12 | M82831 | — | — | 3.38 (5.42)** |
| SPP-1 | X13986 | — | — | 210.00 (187.5)** |

Values shown in parentheses for RETN, MMP-12 and SPP-1 represent fold changes determined by TaqMan in a conformational study. The completed data set, including both up and down-regulated genes can be found in the supplement.
*Normalized to 3 week timepoint.
**TaqMan results in parentheses

TABLE 2
The following risk factors were compared between the normal and
premature (P)CAD individuals making up this population. There
were no significant differences in these traditional risk factors
between the normal and PCAD groups with the exception of age.

| | Normal | PCAD | p value |
|---|---|---|---|
| N | 39 | 38 | |
| Sex (M/F) | 26/13 | 27/11 | |
| Age (yrs) | 57.9 ± 1.4 | 46.2 ± 1.4 | <0.0001 |
| BMI (kg/m²) | 33.9 ± 1.3 | 31.3 ± 1.2 | 0.148 |
| Diabetes | 7/38 | 12/38 | 0.261 |
| Glucose (mg/dl) | 120.2 ± 10.7 | 120.0 ± 8.1 | 0.988 |
| HDL (mg/dl) | 48.9 ± 2.8 | 45.1 ± 2.1 | 0.274 |
| LDL (mg/dl) | 105.8 ± 5.9 | 100.5 ± 9.2 | 0.631 |
| Triglycerides (mg/dl) | 130.2 ± 10.7 | 178.1 ± 29.5 | 0.137 |
| Total Cholesterol (mg/dl) | 183.4 ± 6.4 | 169.4 ± 11.6 | 0.297 |
| Resistin (ng/ml) | 10.7 ± 0.9 | 16.5 ± 1.9 | 0.009 |

Normal individuals were not selected unless they were over the age of 45 years and found (by angiography) to have completely normal coronary arteries, while PCAD individuals were younger than 45 years at the time of diagnosis (either by angiography or documented MI). In this dataset resistin levels were found to be significantly different between the normal and PCAD individuals (p = 0.009
Data are mean ± SE. p values are by student t test. Glucose measurements are non-fasting.

TABLE 3
Correlations of the following risk factors
with premature coronary artery disease

| | Odds Ratio | Confidence Interval | p value |
|---|---|---|---|
| glucose | 1.00 | 0.991-1.009 | 0.9884 |
| HDL | 0.979 | 0.940-1.019 | 0.2957 |
| LDL | 1.006 | 0.990-1.022 | 0.4720 |
| Triglycerides | 1.004 | 0.999-1.010 | 0.1273 |
| Cholesterol | 0.998 | 0.985-1.012 | 0.8275 |
| Resistin | 1.075 | 1.014-1.141 | 0.0155* |

*This finding represents an increase risk of premature coronary artery disease of 1.075 for every increase of 1 ng/ml unit of resistin.

TABLE 4
Correlations of the following risk factors with resistin levels

| | r value | p value |
|---|---|---|
| HDL | −0.113 | 0.39 |
| LDL | 0.081 | 0.58 |
| Total Cholesterol | 0.018 | 0.89 |
| Triglycerides | 0.012 | 0.93 |
| BMI | 0.019 | 0.88 |

### DISCUSSION OF EXPERIMENTAL RESULTS

In the present investigation applicants conducted a time course analysis of gene expression in the aortas of ApoE−/− mice, using Affymetrix GeneChips. Applicants found that, among numerous pro-atherosclerotic genes, resistin mRNA levels steadily increased over time as the lesions increased in size. Applicants confirmed these findings using TaqMan PCR, and determined by immunohistochemical staining that resistin protein is indeed present in atherosclerotic lesions in both mice and humans. Aortic sections taken from wild-type C57BL/6J mice, with no evidence of atherosclerotic lesions, showed no staining for resistin. Further comparison of aortic resistin mRNA levels in a separate group of apoE−/− and C57BL/6J wild-type mice using TaqMan, demonstrated that although resistin, mRNA levels do increase with age in both strains, the atherosclerotic apoE−/− mice have significantly higher levels of resistin mRNA at all time points, compared to the wild-type mice. Further support for a pro-atherosclerotic role of resistin is evidenced by the fact that apoE−/− mice have elevated serum resistin levels compared to wild-type C57BL/6J mice at the same age.

Applicants have shown an association between resistin mRNA and protein levels and the development of atherosclerosis. Applicants demonstrated that recombinant murine resistin upregulates both MCP-1 and sVCAM-1 expression in murine aortic endothelial cells, providing further support for a possible mechanism by which resistin could contribute to the atherogenic process. Applicants determined that the average serum level of resistin in 16 week C57BL/6J apoE−/− mice was 25.4 ng/ml. Although this concentration is below that which was found to have a significant effect on endothelial cell production of MCP-1 and sVCAM-1 (50-100 ng/ml), the average serum level is likely to be lower than the levels that may be present locally in the vessel wall, or in areas of atherosclerotic lesions.

In our study, applicants examined resistin levels in a group of individuals diagnosed by angiography as either having PCAD or normal coronary arteries. Importantly, applicants found that elevated levels of plasma resistin are associated with premature coronary artery disease. There were no significant differences in risk factors, including the incidence of diabetes, between the individuals and controls, with the exception of age Due to our selection criteria, the normal control individuals were older than the PCAD individuals, as they were older than 45 years of age, with angiographically normal coronary arteries, while the PCAD individuals were less than 45 years of age at the time of MI, or diagnosis by angiography. In this population it was determined that individuals with higher levels of resistin (ng/ml) are more likely to have PCAD; specifically, there was an increased risk of PCAD of 80% for every increase of 10 ng/ml of plasma resistin. Applicants found no correlations between resistin levels and HDL, LDL, total cholesterol, triglycerides, or BMI. In our population, there was a trend for diabetics to have higher plasma levels of resistin, but this difference was not statistically significant (15.51 ng/ml vs. 13.09 ng/ml, p=0.361).

We claim:

1. A method of diagnosing or aiding in the diagnosis of a vascular condition in an individual, the method comprising
(a) determining the level of a resistin gene product in a biological sample obtained from the individual; and
(b) comparing the level with a control level, wherein if the level determined in (a) is greater than the control level, the individual is diagnosed as having the vascular condition.

2. The method of claim 1, wherein the biological sample is selected from the group consisting of whole blood, serum and plasma.

3. The method of claim 1, wherein the resistin gene product is a resistin polypeptide.

4. The method of claim 3, wherein the resistin polypeptide is a monomer, homodimer, homotrimer or homohexamer.

5. The method of claim 1, wherein determining the level of a resistin gene product in a biological sample comprises determining the level of a resistin polypeptide having a post-translational modification.

6. The method of claim 1, wherein the resistin gene product is a resistin mRNA.

7. The method of claim 1, wherein determining the level of the resistin gene product in the biological sample comprises determining the bioactivity of a resistin polypeptide in the sample.

8. The method of claim 1, wherein the control level is the average level of the resistin gene product in a population of individuals afflicted with a vascular condition.

9. The method of claim 1, wherein the control level is an average level of the resistin gene product in a population of one or more individuals afflicted with premature coronary artery disease.

10. The method of claim 1, wherein the vascular condition is selected from the group consisting of atherosclerosis, premature coronary artery disease (PCAD) and coronary artery disease (CAD).

11. The method of claim 10, wherein atherosclerosis is selected from cardiovascular atherosclerosis, cerebrovascular atherosclerosis, peripheral vessel atherosclerosis and coronary heart atherosclerosis.

12. The method of claim 1, wherein the vascular condition is a vascular event.

13. The method of claim 12, wherein the vascular event is selected from the group consisting of stroke, unstable angina, tachycardia, vasodilatation, palpitations, syncope heart stroke, clots, unstable angina, cardiac arrest, myocardial infarction, coronary death, non-fatal myocardial infarction, deep venous thrombosis, pulmonary embolism and transient ischemic attack.

14. The method of claim 1, wherein step (a) uses an immunological assay to determine the level of the resistin gene product.

15. The method of claim 14, wherein the immunological assay is a sandwich assay.

16. The method of claim 14, wherein the immunological assay uses a monoclonal antibody having a high affinity for resistin.

17. The method of claim 1, further comprising determining the level of at least one additional gene product in the sample, wherein the additional gene product is selected from the group consisting: Annexin V, B-type natriuretic peptide (BNP), enolase, Troponin T (TnI), cardiac-troponin T, Creatine kinase (CK), Glycogen phosphorylase (GP), Heart-type fatty acid binding protein (H-FABP), Phosphoglyceric acid mutase (PGAM) S-100, soluble tumor necrosis factor-α receptor-2, interleukin-6, and lipoprotein-associated phospholipase A2, C-reactive protein (CRP), Creatine Kinase with Muscle and/or Brain subunits (CKMB), thrombin anti-thrombin (TAT), soluble fibrin monomer (SFM), fibrin peptide A (FPA), myoglobin, thrombin precursor protein (TPP), platelet monocyte aggregate (PMA), troponin, homocysteine, myeloperoxidase (MPO), anti-HSP60 antibodies and HSP70.

18. The method of claim 1, further comprising determining the level of at least one additional gene product in the sample, wherein the additional gene product is a marker which is indicative of atherosclerotic plaque rupture and selected from the group consisting of: human neutrophil elastase, inducible nitric oxide synthase, lysophosphatidic acid, malondialdehyde-modified low density lipoprotein, matrix metalloproteinase-1, matrix metalloproteinase-2, matrix metalloproteinase-3, matrix metalloproteinase-9, myeloperoxidase (MPO) and anti-HSP60 antibodies.

19. The method of claim 1, further comprising determining the level of at least one additional gene product in the sample, wherein the additional gene product is a marker which is indicative of coagulation and selected from the group consisting of: β-thromboglobulin, D-dimer, fibrinopeptide A, platelet-derived growth factor, plasmin-α-2-anti-plasmin complex, platelet factor 4, prothrombin fragment 1+2, P-selectin, thrombin-antithrombin HI complex, thrombus precursor protein, tissue factor and von Willebrand factor.

20. The method of claim 1, further comprising determining the level of at least one additional gene product in the sample selected from the group consisting of adiponectin, leptin and adrenomedullin.

21. A method for predicting the likelihood that an individual will develop a vascular condition, the method comprising
(a) determining the level of a resistin gene product in a biological sample obtained from the individual; and
(b) comparing the level with a control level, wherein if the level determined in (a) is greater than the control level, the individual is said to have increased likelihood of experiencing the vascular event.

22. The method of claim 21, wherein the biological sample is selected from the group consisting of whole blood, serum and plasma.

23. The method of claim 21, wherein the resistin gene product is a resistin polypeptide.

24. The method of claim 23, wherein the resistin polypeptide is a monomer, homodimer, homotrimer or homohexamer.

25. The method of claim 21, wherein determining the level of a resistin gene product in a biological sample comprises determining the level of a resistin polypeptide having a post-translational modification.

26. The method of claim 21, wherein the resistin gene product is a resistin mRNA.

27. The method of claim 21, wherein determining the level of the resistin gene product in the biological sample comprises determining the bioactivity of a resistin polypeptide in the sample.

28. The method of claim 21, wherein the control level is the average level of the resistin gene product in a population of individuals afflicted with a vascular condition.

29. The method of claim 21, wherein the control level is an average level of the resistin gene product in a population of one or more individuals afflicted with premature coronary artery disease.

30. The method of claim 21, wherein the vascular condition is selected from the group consisting of atherosclerosis, premature coronary artery disease (PCAD) and coronary artery disease (CAD).

31. The method of claim 30, wherein atherosclerosis is selected from cardiovascular atherosclerosis, cerebrovascular atherosclerosis, peripheral vessel atherosclerosis and coronary heart atherosclerosis.

32. The method of claim 21, wherein the vascular condition is a vascular event.

33. The method of claim 32, wherein the vascular event is selected from the group consisting of stroke, unstable angina, tachycardia, vasodilatation, palpitations, syncope heart stroke, clots, unstable angina, cardiac arrest, myocardial infarction, coronary death, non-fatal myocardial infarction, deep venous thrombosis, pulmonary embolism and transient ischemic attack.

34. The method of claim 21, wherein step (a) uses an immunological assay to determine the level of the resistin gene product.

35. The method of claim 34, wherein the immunological assay is a sandwich assay.

36. The method of claim 34, wherein the immunological assay uses a monoclonal antibody having a high affinity for resistin.

37. The method of claim 21, further comprising determining the level of at least one additional gene product in the sample, wherein the additional gene product is selected from the group consisting: Annexin V, B-type natriuretic peptide (BNP), enolase, Troponin I (TnI), cardiac-troponin T, Creatine kinase (CK), Glycogen phosphorylase (GP), Heart-type fatty acid binding protein (H-FABP), Phosphoglyceric acid mutase (PGAM) S-100, soluble tumor necrosis factor-α receptor-2, interleukin-6, and lipoprotein-associated phospholipase A2, C-reactive protein (CRP), Creatine Kinase with Muscle and/or Brain subunits (CKMB), thrombin anti-thrombin (TAT), soluble fibrin monomer (SFM), fibrin peptide A (FPA), myoglobin, thrombin precursor protein (TPP), platelet monocyte aggregate (PMA), troponin, homocysteine, myeloperoxidase (MPO), anti-HSP60 antibodies and HSP70.

38. The method of claim 21, further comprising determining the level of at least one additional gene product in the sample, wherein the additional gene product is a marker which is indicative of atherosclerotic plaque rupture and selected from the group consisting of: human neutrophil elastase, inducible nitric oxide synthase, lysophosphatidic acid, malondialdehyde-modified low density lipoprotein, matrix metalloproteinase-1, matrix metalloproteinase-2, matrix metalloproteinase-3, matrix metalloproteinase-9, myeloperoxidase (MPO) and anti-HSP60 antibodies.

39. The method of claim 21, further comprising determining the level of at least one additional gene product in the sample, wherein the additional gene product is a marker which is indicative of coagulation and selected from the group consisting of: β-thromboglobulin, D-dimer, fibrinopeptide A, platelet-derived growth factor, plasmin-α-2-anti-plasmin complex, platelet factor 4, prothrombin fragment 1+2, P-selectin, thrombin-antithrombin III complex, thrombus precursor protein, tissue factor and von Willebrand factor.

40. The method of claim 21, further comprising determining the level of at least one additional gene product in the sample selected from the group consisting of adiponectin, leptin and adrenomedullin.
